(19) United States
(12) Patent Application Publication
(10) Pub. No.: US 2011/0184307 A1
(43) Pub. Date: Jul. 28, 2011

(54) SYSTEM FOR SYNCHRONISING EEG WITH AUXILIARY OUTPUT, IN PARTICULAR VIDEO

(75) Inventor: David Keith Hulin, Surrey (GB)

(73) Assignee: LIFELINES LIMITED, Hampshire (GB)

(21) Appl. No.: 13/055,310

(22) PCT Filed: Jul. 22, 2009
(86) PCT No.: PCT/GB2009/001820

§ 371 Date: Apr. 15, 2011

(30) Foreign Application Priority Data
Jul. 24, 2008 (GB) 0813534.5

(51) Int. Cl. A61B 5/0476

(52) U.S. Cl. 600/544

(57) ABSTRACT

A system for monitoring a patient's EEG output comprises an EEG recorder operative to generate an EEG output indicative of electrical activity produced by the brain of a patient, and an auxiliary recorder operative to generate an auxiliary output indicative of another characteristic of the patient. The auxiliary recorder may comprise a video recorder. An electronic data processor is operative to receive the EEG recorder output and the auxiliary output, the system being operative to generate synchronisation data indicative of when the EEG output and when the auxiliary output occurred with reference to a datum signal. The electronic processor is operative to process the synchronisation data to subsequently synchronise the playback of the EEG output with the auxiliary output. A compensation factor accounts for transmission latency.

The present invention relates to a system for monitoring a patient's EEG output and particularly but not exclusively relates to such a system for monitoring the EEG output of a patient outside of hospital, for example, when the patient is at home.

During EEG studies wherein a patient's EEG output is monitored, it is sometimes the case that a video output is simultaneously made of the patient. The video output can allow a more useful/accurate diagnosis to be made of the patient's condition and medical health. To be useful, the video output ideally needs to be synchronised to the EEG output to the nearest frame, thus allowing accurate playback of the video output frame by frame with the associated EEG output.

A problem arises when attempting to synchronise separate devices in that the EEG recorder, the PC and the video camera and hardware are all unsynchronised sources of data. For example, video cameras can drift many minutes over a 24 hour period.

According to a first aspect of the invention there is provided a system for monitoring a patient's EEG output comprising an EEG recorder operative to generate an EEG output indicative of electrical activity produced by the brain of a patient, an auxiliary recorder operative to generate an auxiliary output indicative of another characteristic of the patient, and an electronic data processor operative to receive the EEG recorder output and the auxiliary output, the system being operative to generate synchronisation data indicative of when the EEG output and when the auxiliary output occurred with reference to a datum signal, the electronic processor being operative to process the synchronisation data to subsequently synchronise the playback of the EEG output with the auxiliary output, wherein the electronic data processor is operative to apply a compensation factor to at least one of the EEG and auxiliary outputs that accounts for the latency of transmission of the at least one output from the recorder to the electronic data processor, the latency being a value indicative of the time delay between the respective recorder transmitting the output and the electronic data processor receiving the output

Preferably the EEG recorder is operative to receive an initiation signal from the electronic data processor, the initiation signal including a datum signal.

Preferably the synchronisation data includes data relating the initiation of EEG output to the datum signal.

Preferably the synchronisation data includes data relating the duration of EEG output to the datum signal.

Preferably the synchronisation data is transmitted to the electronic data processor from the EEG recorder.

Preferably the auxiliary recorder is operative to receive an initiation signal from the electronic data processor, the initiation signal including a datum signal.

Preferably the synchronisation data includes data relating the initiation of auxiliary output to the datum signal.

Preferably the synchronisation data includes data relating the duration of auxiliary output to the datum signal.

Preferably the synchronisation data is transmitted to the electronic data processor from the auxiliary recorder.

Preferably the auxiliary recorder comprises a video recorder.

Preferably the electronic data processor is controlled to generate a synchronisation timer operative to generate periodic recording initiation signals.

Preferably the periodic recording initiation signals are calculated with reference to the datum signal.

Preferably the electronic data processor is controlled to generate periodic synchronisation files in which a time identifier is attached to the EEG output and the auxiliary output that occur at each periodic recording initiation signal. Each periodic synchronisation file therefore preferably includes a time identifier associated with a particular segment of EEG and auxiliary recorder output.

Preferably the time identifier references the EEG output and the auxiliary output to the datum signal.

Preferably at least one of the EEG recorder and the auxiliary recorder comprise a memory device operative to store the respective output on the recorder at source.

Preferably the electronic data processor is operative to automatically synchronise the EEG output with the auxiliary output on playback of either of the outputs.

Preferably the system comprises transmission means.

The transmission means may comprise a wired or a wireless transmitter.

Where the transmission means comprises a wireless transmitter, and in use of the system the transmitter moves out of transmission range with the electric data processor, the system is operative to automatically resynchronise the auxiliary output with the EEG recorder output when the transmitter moves back into transmission range.

The transmission means may be integral with the EEG recorder or may comprise a separate adaptor removably connectable to the EEG recorder.

According to a second aspect of the invention there is provided an EEG recorder for use with a system for monitoring a patient's EEG output, the system comprising an electronic data processor operative to receive' an auxiliary output generated by an auxiliary recorder, the EEG recorder being operative to generate an EEG output indicative of electrical activity produced by the brain of a patient, the EEG recorder further comprising transmission means to transmit the EEG output to the electronic data processor with synchronisation data indicative of when the EEG output occurred with reference to a datum signal, the synchronisation data being capable of being processed by the electronic data processor to enable the electronic data processor to subsequently synchronise playback of the EEG output with the auxiliary output.

According to a third aspect of the invention there is provided an electronic data processor operative to receive an EEG output indicative of electrical activity produced by the brain of a patient, and to receive an auxiliary output indicative of another characteristic of the patient from an auxiliary recorder, the electronic data processor being controlled to process synchronisation data indicative of when the EEG output and the auxiliary output occurred so as to subsequently synchronise the playback of the EEG output with the auxiliary recorder output, wherein the electronic data processor is operative to apply a compensation factor to at least one of the EEG and auxiliary outputs that accounts for the latency of transmission of the at least one output from the recorder to the electronic data processor, the latency being a value indicative of the time delay between the respective recorder transmitting the output and the electronic data processor receiving the output.

Other aspects of the present invention may include any combination of the features or limitations referred to herein.

The present invention may be carried into practice in various ways, but embodiments will now be described by way of example only with reference to the accompanying drawings in which:

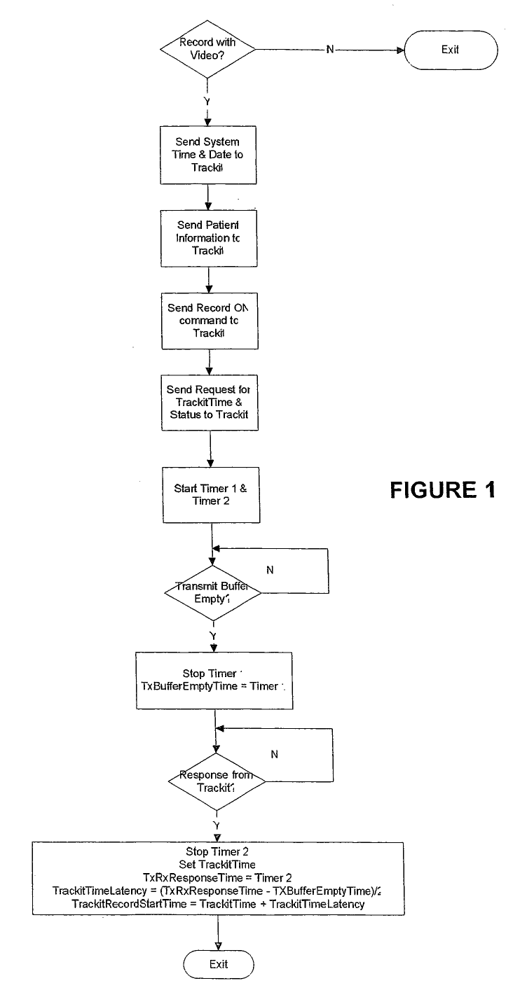

FIG. 1 is a flow diagram illustrating process steps involved in starting recording using a system in accordance with the present invention;

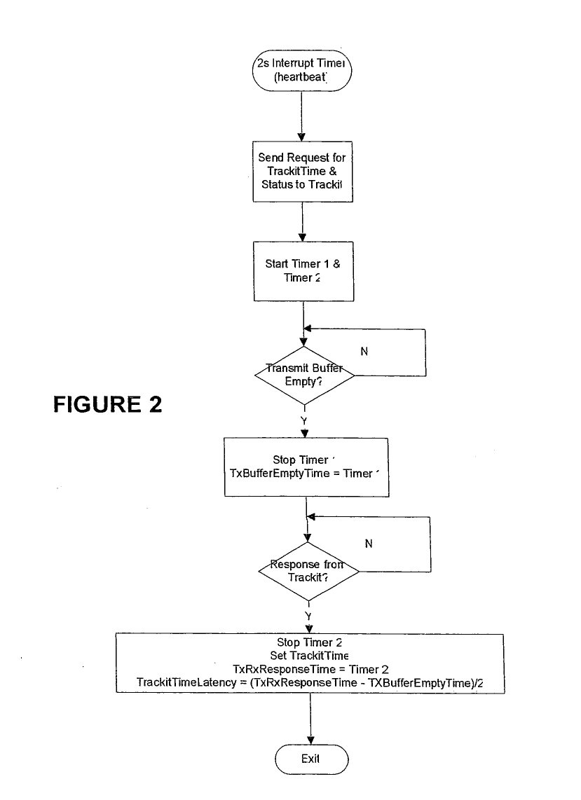

FIG. 2 is a flow diagram illustrating process steps involved with a heartbeat timer comprising part of a system in accordance with the present invention; and

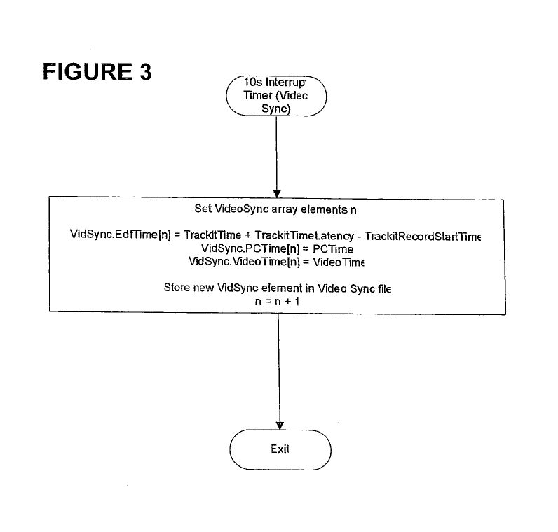

FIG. 3 is a flow diagram illustrating process steps involved in a synchronisation timer comprising part of a system in accordance with the present invention.

### SYSTEM OVERVIEW

A system for monitoring a patient's EEG comprises three primary components: an EEG recorder, an auxiliary recorder which in this example comprises a video recorder, and an electronic data processor which in this example comprises a PC.

The EEG recorder comprises an EEG data processor contained in a hand portable housing that can be carried on the patient as they move around, for example in a clothing pocket or on a belt. The EEG recorder further comprises EEG sensors adapted to be secured to the head of the user so as to provide an EEG output indicative of the electrical activity produced by the brain of the patient. The EEG recorder preferably comprises a memory device operative to store the EEG output at source prior to onward transfer to the PC. The EEG recorder thus comprises a single unit that combines a EEG data source and a EEG data storage device. This can help to reduce latency delays between the EEG recorder and the PC. Alternatively the EEG output may be immediately transmitted to the PC.

Transmission of the EEG output to the PC is enabled via transmission means which may be wired or wireless. If wired, the transmission means may comprise a data cable of any desired type including, for example, a fibre optic cable. If wireless, the transmission means may comprise any desired wireless transmitter including for example a Bluetooth® transmitter. The wireless transmitter may be integral with the EEG recorder or comprise an adapter removably connectable to the EEG recorder.

The PC is also operative to receive an auxiliary output which in this example comprises a video output of the patient as obtained from the video recorder which again preferably comprises a memory device for storing the video output prior to subsequent transmission to the PC. The video recorder thus also comprises a single unit that combines the video data source and a video data storage device.

The PC, the EEG recorder and the video recorder are controlled via software on the PC. It is envisaged that the control functions provided by the software could alternatively be provided by hardware on the PC.

The software controls the PC to receive the outputs from the EEG recorder and the video recorder, to synchronise those outputs such that an output from the EEG at a given time is synchronise'd with the output of the video recorder at that time, and to subsequently enable the two outputs to be played back in synchronisation such that EEG events occurring at a given time can be viewed simultaneously with video events occurring at the same time. This simultaneous synchronised playback of the two data sources enables potentially useful correlations to be made between a patient's brain activity and other activity, such as physical movement for example.

The software uses the PC clock to generate a datum signal indicative of the date and time of the system and which operates independently of the EEG recorder and the video recorder.

On initiation of the system, the datum signal is transmitted to the EEG recorder, together with an identifier identifying the patient in question. EEG recording then begins. The exact time that the EEG recording begin is noted with reference to the datum signal and thereafter the elapsed EEG recording time can be periodically calculated using a heartbeat timer controlled by the software. So the EEG recording start time, and the EEG recording elapsed time, can be calculated with reference to the datum signal, that is, with reference to the PC time. This means that the position of the EEG output is known with reference to a given datum time, it being irrelevant whether or not this datum time corresponds to the actual time, or the time indicated on either the EEG recorder or the video recorder.

The EEG output is preferably stored on the EEG recorder and then subsequently transferred to the PC, or transmitted in real-time for storage on the PC. Either way, the EEG output includes synchronisation data indicative of the time/date of the output with reference to the PC time/date. When subsequently transferred, this may be achieved by unplugging the EEG recorder's flash memory card and plugging the card into the PC.

The video output is similarly obtained, stored and transmitted to the PC and the software generates further synchronisation data indicative of relating the start time of the video output, and the elapsed duration of the video output, with reference to the PC time.

The software includes a synchronisation timer that is used by the software to periodically record the synchronisation data relating when the EEG output and video output occurred to the PC time in periodic synchronisation files. Effectively the synchronisation timer is used to electronically time/date stamp batches of EEG and video files, each time/date stamp referencing the batch of files to the PC time. So regardless of the accuracy or actual value of the PC time, each batch of files can be correlated.

The software generates folders of synchronisation files each of which includes three strands of data: the PC time at which the synchronisation file was created; the time elapsed between the exact PC time that the EEG output began and the PC time at which the synchronisation file in question was created; and the time elapsed between the exact PC time that the video output began and the PC time at which the synchronisation file in question was created.

The software is also operative to calculate the latency between the EEG recorder and the PC, and between the video recorder and the PC, the latency being the difference between the data transmission time and the data reception time. This latency value is used to generate a compensation factor to any time data received from the EEG recorder or the video recorder to allow for the possibility of a transmission delay on data from these recorders.

The following describes in more detail the system functionality outlined above.

### Files Created

1. The following files are created during acquire:
filename.edf (the EEG file, either recorded on the EEG recorder and subsequently transferred, or immediately transmitted and stored on the PC)
filename.tev (the event file)
filename_*.avi or filename*.wmv (the video recorder file)
filename_*.tvs (the synchronisation file)

All these files for the particular patient and the current study reside in the same folder on the PC. Note that “*” means any time/date stamp. The software automatically appends a unique time/date stamp to the video file name whenever a video recording is started.

2. The video file is either.avi extension or .wmv extension. The encoding can be any available on the PC at the time, eg. Intel Indeo, WMVideo8/9, MPEG, MJPEG, DV Video, Windows Media Profiles, Microsoft H.263 etc.

3. If recorded with audio, the encoding can be any available on the PC at the time, eg. PCM, ADPCM, DV Audio, Windows Media Audio V1/2, MPEG etc. Mono or Stereo.

4. There can be many video recorder files associated with the current study. These all have the same file name root but have different time/date stamps. Within the software there are options to record the video file split into sections either of a certain size or certain time. This is more convenient than having a single large file.

5. There is only a single EEG file with the particular filename root in the folder. There can be different EEG files with different root names and their associated video files all in the same folder or each study can be stored in separate folders.

6. The time/date stamp format is as follows: filename_yymmddhhmmss. Windows Explorer will automatically sort these into chronological order (whatever the file create, modify, copy dates show).

7. The video and EEG output playback will be in synchronisation (move together) if their respective times and dates overlap and the patient name and recording ID are identical. There will be no synchronisation if the synchronisation file is missing or the times do not coincide.

### Synchronisation File

The synchronisation file (extension “tvs”) contains data to allow the two data streams (from the EEG recorder and the video recorder) to be synchronised. The contents of the file is defined as follows:

### 1. Header A

All fields in Header A are strings. The size of Header A is 256 bytes.

| Byte Offset | Field name | Type | Length | Description |
|---|---|---|---|---|
| 0 | VidSyncVersion | string | 8 | File version = “tvs0” |
| 8 | VidSyncPatientId | string | 80 | The PatientID field is the same as |
| | | | | in the edf and tev files |
| 88 | VidSyncRecordId | string | 80 | The RecordID field is the same as |
| | | | | in the edf and tev files |
| 168 | VidSyncStartDate | string | 8 | The start date of the recording. |
| | | | | Format is dd.mm.yy |
| 176 | VidSyncStartTime | string | 8 | The start time of the recording. |
| | | | | Format is hh.mm.ss |
| 184 | VidSyncHeaderSize | string | 8 | The total size of Header A and |
| | | | | Header B in bytes = “512” (unless |
| | | | | more than 16 video event types are |
| | | | | required - refer below) |
| 192 | VidSyncTotalEvents | string | 8 | The total number of |
| | | | | events/syncpoints stored in the |
| | | | | VidSyncList section of the file |
| 200 | VidSyncFrameRateX1000 | string | 8 | Video frame rate multiplied by |
| | | | | 1000 (mHz) |
| 208 | VidSyncDroppedFrames | string | 8 | Total number of dropped frames in |
| | | | | the video recording |
| 216 | VidSyncLengthms | string | 10 | Total length of video recording in |
| | | | | ms |
| 226 | VidSyncLengthFrames | string | 8 | Total length of video recording in |
| | | | | frames |
| 234 | VidSyncManualOffsetms | string | 8 | Manual video offset in ms. Allows |
| | | | | for storage of a manual offset |
| | | | | setting applied during playback |
| 242 | VidSyncReserved | string | 14 | Unused |

All strings are left-justified and padded with spaces.

### 2. Header B

All fields in Header B are strings. The size of Header B is 16×16 byte entries=256 bytes. Each 16 byte entry is a description of a type of video event which is referenced in the VideoSyncList portion of the file by a negative value of −1 to −15 respectively. If more video event types are required then another Header B type record is contained in the file and the VidSyncHeaderSize entry is adjusted appropriately.

| Byte Offset | Field name.element | Type | Length | Description |
|---|---|---|---|---|
| 0 | VidSyncKey0.0 | string | 16 | Description for Video Event 0 |
| | | | | (“No Event”) |
| 16 | VidSyncKey0.1 | string | i6 | Description for Video Event 1 |
| 32 | VidSyncKey0.2 | string | 16 | Description for Video Event 2 |
| 48 | VidSyncKey0.3 | string | 16 | Description for Video Event 3 |
| 64 | VidSyncKey0.4 | string | 16 | Description for Video Event 4 |
| 80 | VidSyncKey0.5 | string | 16 | Description for Video Events |
| 96 | VidSyncKey0.6 | string | 16 | Description for Video Event 6 |
| 112 | VidSyncKey0.7 | string | 16 | Description for Video Event 7 |
| 128 | VidSyncKey0.8 | string | 16 | Description for Video Event 8 |
| 144 | VidSyncKey0.9 | string | 16 | Description for Video Event 9 |
| 160 | VidSyncKey0.10 | string | 16 | Description for Video Event 10 |
| 176 | VidSyncKey0.11 | string | 16 | Description for Video Event 11 |
| 192 | VidSyncKey0.12 | string | 16 | Description for Video Event 12 |
| 208 | VidSyncKey0.13 | string | 16 | Description for Video Event 13 |
| 224 | VidSyncKey0.14 | string | 16 | Description for Video Event 14 |
| 240 | VidSyncKey0.15 | string | 16 | Description for Video Event 15 |

All strings are left-justified and padded with spaces.

### 3. VideoSyncList

All data in this section and for the remainder of the file are groups of 3 long (32 bit) integers. These periodic recording initiation signals or ‘Sync points’ are used to periodically record, at specific times of the day with reference to the PC time, the corresponding EEG recording time and the video recording time. In this way synchronisation data is available relating all three variable clock sources—the PC time, the EEG time and the video time. Alternatively, an entry can record a video event. There are as many of these triplet entries as is given in VidSyncTotalEvents in the Header.

| Byte | Field | | Length | |
|---|---|---|---|---|
| Offset | Name | Type | bytes | Description |
| 0 | Edfms | Long | 4 | Elapsed EEG recording time in |
| | | | | ms |
| 4 | PCTimems | Long | 4 | PC time in ms since midnight on |
| | | | | the day the recording started. |
| 8 | Videoms | Long | 4 | Elapsed video recording time in |
| | | | | ms |

The sync points are nominally at 30 second intervals throughout the recording, although at the beginning of the recording there are three Sync Points at 10 second intervals. The exact timing is unimportant because the data entry includes the actual PC time of the sync point.

If both of Edfms and Videoms is >0 then the timing data for that particular sync point relating the EEG and video and PC time is valid.

If either of Edfms or Videoms is =0 then the timing data for that particular sync point for either the EEG or video was not available at the time. This does mean, however, that the other parameter which is >0 is still valid and can be used to relate to PC time.

If either of Edfms or Videoms is <0 then this sync point relates to a video event. Each value from −1 to −15 specifies one of the event types 1 to 15 respectively in Header B.

If recording over midnight, the PCTimems value will not rollover to 0. Instead it will be pc time+86400000, where 86400000 is the number of milliseconds in 24 hours. This occurs for the current, contiguous file that overlaps midnight. For subsequent files in the set, the pc time roll over does occur because the file time/date stamp has already rolled over to the next day.

The video file start time is given by:
If VideoSyncFile exists then video file start time=PCTimemsₙ−Videomsₙ (where n=the first syncpoint which has Videoms>0).
If VideoSyncFile missing then video file start time=Video time/date stamp

The video file start date=Time/date stamp

The EEG file start time is given by:
If VideoSyncFile exists then EEG file start time=PCTimemsₙ−Edfmsₙ (where n=the first syncpoint which has Edfms>0)
If VideoSyncFile missing then Edf file start time=Edf Header time/date

The EEG file start date=Edf Header time/date or Time/date stamp (the dates should be the same).

The system software allows the recording of video files synchronised to the EEG edf file. Both EEG and video recordings can be stored on the PC having been transmitted with or without the wireless Bluetooth link. Alternatively, the EEG recording may be stored on the EEG recorder with the synchronised video recording stored on the PC, the video recording again having been transmitted from the video recorder either with or without the wireless Bluetooth link.

During playback, the software in operative to control the PC to enable play backwards or forwards either the EEG file with video automatically tracking, or video with EEG automatically tracking. There is provision for variable speed playback including EEG paged mode. Also provided is a single frame mode wherein the EEG or video recording or both is/are stepped forwards or backwards.

### Playback

Playback is available only when the EEG recorder is offline

Open files for playback opens the following files:
filename.edf (the eeg file, either recorded on the Trackit and transferred or on the PC)
filename.tev (the event file)
filename_*.avi or filename^{t}.wmv (the video file)
filename_{—}^{t}.tvs (the video sync file)

All these files for the particular patient and the current study are stored in the same folder on the PC. Note that ‘*’ means any time/date stamp. The software automatically appends a unique time/date stamp to the video file name whenever a video recording is started.

There can be many video files associated with the current study. These all have the same file name root but have different time/date stamps. Within the software there are options to record the video file split into sections a certain size or certain time. This is more convenient than having a single large file.

The software automatically opens the first video file in the playback window which has the closest start time to the EEG recording start time. All other video files associated with the study are listed in properties underneath the playback window.

There is only a single EEG (edf) file with the particular filename root in the folder. There can be different EEG (edf) files with different root names and their associated video files all in the same folder. It is also possible to split each study into a separate folder.

The time/date stamp format is as follows: filename_yymmddhhmmss. Windows Explorer will automatically sort these into chronological order (whatever the file create, modify, copy dates show).

The video and edf playback will be in synchronisation (move together) if their respective times and dates overlap and the patient name and recording ID are identical. There will be no synchronisation if the video sync file is missing or the times do not coincide.

Even if synchronisation is not possible due to one of the above reasons, it is still possible to playback the video file manually provided the filename root is correct.

Once the playback window is open, any video file anywhere on the PC can be opened from the open file menu.

Right-clicking on a video file in the file list brings up Properties and Delete pop-up menu.

### PC Recording

This is the situation where both the EEG and video outputs are stored on the PC. The EEG recorder is used as a headbox. The link between the PC and the EEG recorder can be wired, or wireless Bluetooth.

At any time during EEG recording on the PC, the video recording can be stopped and restarted after the first recording.

If EEG recording is already in progress when starting a video recording, synchronisation will automatically start at video start.

A video recording cannot be started unless recording is already in progress on the PC or EEG recorder.

If the EEG recorder is offline, a video recording can be started but this will be unsynchronised.

Stop or start EEG recording always stops the video recording.

EEG recorder offline always stops EEG recording and video recording.

If the EEG recorder is in ‘Auto record when Host comms. Lost’ mode, then when the EEG recorder disconnects (or goes out of wireless range) the video recording will stop even though the EEG recorder will start EEG recording. The PC will attempt to automatically reconnect (see below).

Options are provided by the software for setting video file segment lengths in time or Mbytes and also the total recording time limit.

### EEG Recorder Recording

This is the situation where the EEG is recorded on the EEG recorder and the video is recorded on the PC. The link between the EEG recorder and the PC, which can be wired or wireless Bluetooth, maintains the synchronisation between the EEG and the video outputs.

The video recording on the PC can be stopped and restarted at any time after the first recording. If the EEG recorder is online, synchronisation will be maintained.

If the EEG recorder disconnects (or goes out of wireless range), the PC will continue the video recording and will automatically attempt to reconnect at 10 second intervals. If reconnection is achieved and a video recording is in progress and the EEG recorder is recording and its filename, patient name and ID are the same as for the video file, then video synchronisation will recommence.

Stop EEG recorder recording from PC also stops video recording.

At EEG recorder connect, if it is recording and a video recording is in progress, then synchronisation will start if filename, patient name and ID are all the same.

For playback, the EEG (edf) file and tev file should be copied into the same folder as the video files on the PC.

Video Resume mode retains Patient name and Recording ID after program shut down and PC shut down, so that video recording can continue with automatic connection of the recording EEG recorder. This works if enabled and for a maximum of 24 hours after the program was last shut down after having made at least one video recording.

Options are provided by the software for setting video file segment lengths in time or Mbytes and also total recording time limit.

### Example System Specifications

Support for camera devices that are compatible with WDM/DirectX:
   PCI and PC-Card frame grabbers
   FireWire cameras (DCAM)
   USB/USB 2 cameras
   DV (digital video) devices
Video capture to AVI or WMV file with optional audio
Video compression (codec) support eg:
   WMVideo, WMVideo8 and WMVideo9
   MPEG, MJPEG, DV Video
   Windows Media Profiles
   Intel Indeo video V2.50, R3.2, 5.10 etc
   Microsoft H.261 and H.263, RLE, Video 1
   Any other video codec on the system
Audio compression (codec) support, eg:
   Windows Media Audio Vi and V2
   PCM, ADPCM
   DV Audio
   MPEG
Any other audio codec on the system
Enumeration of all available video capture devices
Variable video frame rate, frame size etc.
Variable audio bit rate, mono/stereo and 8 bit/16 bit
Support for Windows Media Profiles
Live video preview
Custom text caption and time-stamp insertion onto live video
Copy video frame to the clipboard
Save frame as JPG file
Optional time limit and file size limit
Motion detection option
Built-in DirectX media player
Select video input channel to use with multi-input video capture devices
Set video properties (saturation, hue, brightness, etc.)
Select audio input channel to use with multi-input audio devices
Horizontal and/or vertical flipping of the video stream

### EEG Recorder Videometry Recording

FIGS. 1 to 3 illustrate the three processes involved during recording that accomplish synchronisation of the EEG and video outputs:

With reference to FIG. 1, when the user wants to start a recording, this function achieves the following:
Patient Information is sent to the EEG recorder.
The EEG recorder recording is started.
The exact EEG recorder time that the recording starts is calculated. This is as opposed to PC time or Video time. Thereafter, the EEG recorder time can be read periodically (see below) and the elapsed EEG recorder recording time ascertained at any time.
The latency (transmission/reception time) is calculated over the particular wireless or wired communication link being used. This value is used to apply a compensation factor to the time information received from the EEG recorder.

With reference to FIG. 2, an interrupt timer generates a constant 2 s ‘heartbeat’ interrupt signal that repeatedly reads the EEG recorder's current time. As in the above function, the transmit/receive latency is measured and applied as a compensation factor.

If at any time another function requires the EEG recorder time which does not coincide with the 2 s heartbeat interrupt, then the function interpolates since it knows how long has elapsed since the last heartbeat.

With reference to FIG. 3, a synchronisation timer generates a constant 10 s interrupt signal that records a new set of the three items of synchronisation data in the sync file. These are the EEG recorder time, the PC time and the Video time. The exact timing of the 10 s interrupt is unimportant since it is the correlation between the three times at this particular moment that is important, not the time interval since the last sync point was recorded. In practice, after approximately 30 s of recording, the timer interval is increased to 30 s. This reduces the number of sync points to be processed, whilst allowing a very good synchronisation performance, ie no device will drift significantly in 30 s.

It is envisaged that the above described system could synchronise any device output with an EEG output.

1. A system for monitoring a patient's EEG output comprising an EEG recorder operative to generate an EEG output indicative of electrical activity produced by the brain of a patient, an auxiliary recorder operative to generate an auxiliary output indicative of another characteristic of the patient, and an electronic data processor operative to receive the EEG recorder output and the auxiliary output, the system being operative to generate synchronisation data indicative of when the EEG output and when the auxiliary output occurred with reference to a datum signal, the electronic processor being operative to process the synchronisation data to subsequently synchronise the playback of the EEG output with the auxiliary output, wherein the electronic data processor is operative to apply a compensation factor to at least one of the EEG and auxiliary outputs that accounts for the latency of transmission of the at least one output from the recorder to the electronic data processor, the latency being a value indicative of the time delay between the respective recorder transmitting the output and the electronic data processor receiving the output.

2. The system of claim 1 wherein the EEG recorder is operative to receive an initiation signal from the electronic data processor, the initiation signal including the datum signal.

3. The system of claim 2 wherein the synchronisation data includes data relating the initiation of EEG output to the datum signal.

4. The system of claim 2 wherein the synchronisation data includes data relating the duration of EEG output to the datum signal.

5. The system of claim 1 wherein the synchronisation data is transmitted to the electronic data processor from the EEG recorder.

6. The system of claim 1 wherein the auxiliary recorder is operative to receive an initiation signal from the electronic data processor, the initiation signal including a datum signal.

7. The system of claim 6 wherein the synchronisation data includes data relating the initiation of auxiliary output to the datum signal.

8. The system of claim 6 wherein the synchronisation data includes data relating the duration of auxiliary output to the datum signal.

9. The system of claim 1 wherein the synchronisation data is transmitted to the electronic data processor from the auxiliary recorder.

10. The system of claim 1 wherein the auxiliary recorder comprises a video recorder.

11. The system of claim 1 wherein the electronic data processor is controlled to generate a synchronisation timer operative to generate periodic recording initiation signals.

12. The system of claim 11 wherein the periodic recording initiation signals are calculated with reference to the datum signal.

13. The system of claim 11 wherein the electronic data processor is controlled to generate periodic synchronisation files in which a time identifier is attached to the EEG output and the auxiliary output that occur at each periodic recording initiation signal.

14. The system of claim 13 wherein each periodic synchronisation file therefore preferably includes a time identifier associated with a particular segment of EEG and auxiliary recorder output.

15. The system of claim 14 wherein the time identifier references the EEG output and the auxiliary output to the datum signal.

16. The systems of claim 1 wherein, at least one of the EEG recorder and the auxiliary recorder comprise a memory device operative to store the respective output on the recorder at source.

17. The system of claim 1 wherein the electronic data processor is operative to automatically synchronise the EEG output with the auxiliary output on playback of either of the outputs.

18. The system of claim 1 wherein the system comprises transmission means.

19. The system of claim 18 wherein the transmission means comprises a wired transmitter.

20. The system of claim 18 wherein the transmission means comprises a wireless transmitter.

21. The system of claim 20 wherein in use of the system when the transmitter moves out of transmission range with the electric data processor, the system is operative to automatically resynchronise the auxiliary output with the EEG recorder output when the transmitter moves back into transmission range.

22. The system of claim 18 wherein the transmission means is integral with the EEG recorder.

23. The system of claim 18 wherein the transmission means comprises a separate adaptor removably connectable to the EEG recorder.

24. An EEG recorder for use with a system for monitoring a patient's EEG output, the system comprising an electronic data processor operative to receive an auxiliary output generated by an auxiliary recorder, the EEG recorder being operative to generate an EEG output indicative of electrical activity produced by the brain of a patient, the EEG recorder further comprising transmission means to transmit the EEG output to the electronic data processor with synchronisation data indicative of when the EEG output occurred with reference to a datum signal, the synchronisation data being capable of being processed by the electronic data processor to enable the electronic data processor to subsequently synchronise playback of the EEG output with the auxiliary output.

25. An electronic data processor operative to receive an EEG output indicative of electrical activity produced by the brain of a patient, and to receive an auxiliary output indicative of another characteristic of the patient from an auxiliary recorder, the electronic data processor being controlled to process synchronisation data indicative of when the EEG output and the auxiliary output occurred so as to subsequently synchronise the playback of the EEG output with the auxiliary recorder output, wherein the electronic data processor is operative to apply a compensation factor to at least one of the EEG and auxiliary outputs that accounts for the latency of transmission of the at least one output from the recorder to the electronic data processor, the latency being a value indicative of the time delay between the respective recorder transmitting the output and the electronic data processor receiving the output.
